(19) United States
(12) Patent Application Publication
(10) Pub. No.: US 2010/0100038 A1
(43) Pub. Date: Apr. 22, 2010

(54) ELECTRONIC FLOW CONTROL

(75) Inventors: Gregory L. Walker, Carmel, IN (US); Geoff Roper, Zionsville, IN (US)

Correspondence Address: BAKER & DANIELS LLP, 300 NORTH MERIDIAN STREET, SUITE 2700, INDIANAPOLIS, IN, 46204, US

(73) Assignee: Symbios Medical Products, LLC, Indianapolis, IN (US)

(21) Appl. No.: 12/579,651

(22) Filed: Oct. 15, 2009

Related U.S. Application Data
(60) Provisional application No. 61/105,452, filed on Oct. 15, 2008.
(60) Provisional application No. 61/107,780, filed on Oct. 23, 2008.

(51) Int. Cl. A61M 5/168; A61M 5/142; G05D 7/06; G05D 11/13

(52) U.S. Cl. 604/82; 604/151; 700/282

(57) ABSTRACT

An electronic controller is provided for controlling the evacuation of fluid from one or more fluid reservoirs. The controller includes a plurality of sensors that sense environmental factors and the controller adjusts the evacuation of fluid from the bladder(s) using information gathered by the sensors.

### CLAIM FOR PRIORITY

The present application claims priority to U.S. Provisional Application 61/105,452 filed Oct. 15, 2008, and to U.S. Provisional Application 61/107,780 filed Oct. 23, 2008, the disclosures of which are incorporated herein by reference.

### FIELD

The present disclosure relates generally to the administration of liquid agents to patients. More particularly, the present disclosure relates to a device and method that provides for electronic flow control from portable and disposable infusion pumps for various agents and conditions.

### BACKGROUND AND SUMMARY

Drug delivery devices are used to infuse medications or other biologically active substances into human or animal subjects. For administration of selected substances, a substantially constant level of the substance is desired over an extended period. Accordingly, while a large initial bolus dose may be used in conjunction with the extended dose, the initial bolus dose is typically unsuitable for achieving an extended dose by itself. Furthermore, applications arise that require the administration of more than one substance to a particular patient or administration of one substance at multiple locations. Additionally, subsequent bolus doses and dosing regimens are sometimes needed when outside of direct contact with a healthcare provider.

Accordingly, a device and method are provided that allow the administration of one or more substances over defined periods of time and administration of the same substance at multiple locations over defined periods of time.

According to one aspect of the present disclosure, a portable kit for providing pain relief is provided comprising: a catheter for delivering an anesthetic-containing fluid adjacent a wound site in a body; tubing attached to the catheter and having a lumen in fluid communication with a lumen of the catheter; a pump adapted to deliver fluid, the pump including an outlet in fluid communication with the tubing, the pump further including an inlet port for filling a reservoir of the pump; the pump affixable to the body to permit portability; an electronic valve disposed between the catheter and the reservoir in a first flow path; a controller that generates signals to control operation of the electronic valve; and instructions on a controller interpretable medium that when interpreted by the controller cause the controller to be able to selectively place the electronic valve in each of three conditions, the first condition causing the electronic valve to periodically open and close to achieve a first output flow rate through the catheter, a second condition causing the electronic valve to open and close to achieve a second output flow rate, and the third condition causing closure of the electronic valve.

According to another aspect of the present disclosure, a portable kit for providing pain relief is provided comprising: a first pump having a first reservoir that when filled with an anesthetic provides a first pressure biased to evacuate the anesthetic from the first pump; the first pump being constructed such that the first pressure varies based upon the first reservoir experiencing environment induced pressure variations; and an electronic controller that controls the evacuation of anesthetic from the first reservoir to compensate for the variability in the first pressure to achieve a desired output flow that is independent of the environment induced pressure variations experienced by the first reservoir.

According to another aspect of the present disclosure, a portable kit for providing medication to tissue is provided comprising: a pump having a first reservoir, the first reservoir, when filled with a first fluid provides a first pressure biased to evacuate the first fluid from the first reservoir; and an electronic controller that controls the evacuation of fluid from the first reservoir, the electronic controller being able to receive input indicative of the activity of a patient, the electronic controller adjusting an output flow from the first reservoir at least partially based upon the input indicative of the activity of the patient.

According to another aspect of the present disclosure, a portable kit for providing fluid to tissue is provided comprising: a pump having a first reservoir and a second reservoir, an electronic controller that controls the evacuation of fluid from the first and second reservoirs, the electronic controller being able to receive input selected from the group including input indicative of patient status and input indicative of atmospheric status, the electronic controller adjusting output flows from the first and second reservoirs at least partially based upon the received input to achieve a output mixture of the first and second fluids, the mixture having a first ratio of the first fluid to the second fluid.

According to yet another aspect of the present disclosure, a portable kit for providing fluid to tissue is provided comprising: a pump having a first reservoir and a second reservoir, an electronic controller that controls the evacuation of fluid from the first and second reservoirs, the electronic controller being programmable to cause an output flow of the pump to vary as a function of time.

Additional features of the present disclosure will become apparent to those skilled in the art upon consideration of the following detailed description of the presently perceived best mode of carrying out the disclosure.

### BRIEF DESCRIPTION OF THE DRAWINGS

The detailed description of the drawings particularly refers to the accompanying figures in which:

FIG. 1 is a schematic view of a controller and an infusion pump.

### DETAILED DESCRIPTION OF THE DRAWINGS

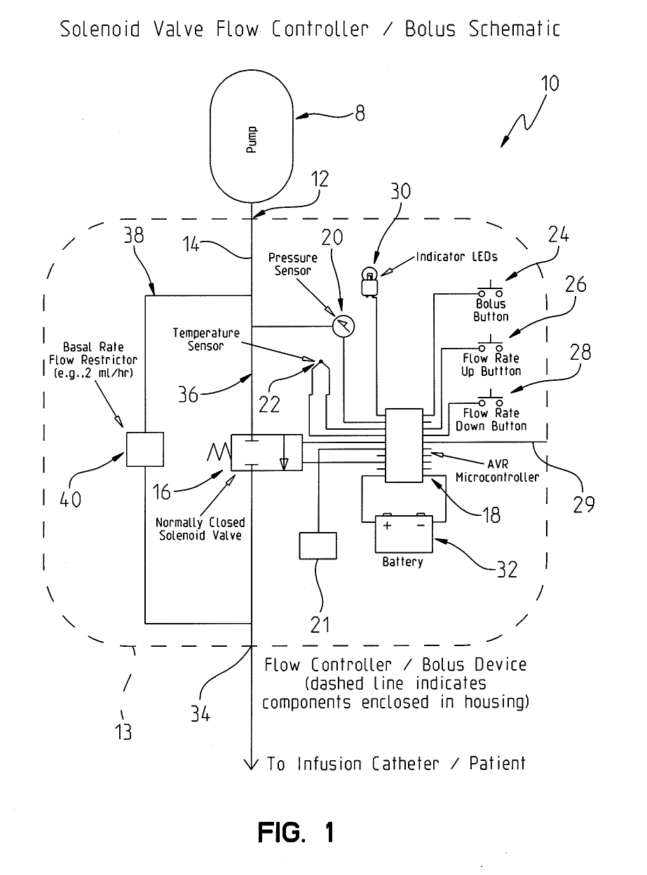

FIG. 1 shows disposable infusion pump 8 coupled to controller 10. Pump 8 is illustratively a GO pump that is commercially available from Symbios Medical Products. Pump 8 is an elastomeric balloon reservoir pump that is non-powered. In other words, pump 8 does not require electrical power to output fluid. Furthermore, pump 8 includes a single fluid containing reservoir. However, other pumps that are powered or that have more than one discreet reservoir therein are also envisioned. Other examples of pumps that are envisioned for use in the present disclosure are those powered by springs and vacuums.

Pump 8 includes a clasp extension (not shown) such that pump 8 may be selectively coupled to a user's belt, pocket, or the like. Elastomeric balloon reservoirs and elastomeric balloon reservoir pumps are described more completely in U.S. Patent Publication No. 2008/0097317-A1, the disclosure of which is incorporated herein by reference.

An output (not shown) from pump 8 is in fluid communication with input 12 of controller 10. Controller 10 includes housing 13, fluid pathway 14, metering solenoid 16, microcontroller 18, sensors 20, 21, 22, buttons 24, 26, 28, electrical I/O port 29, indicators 30, and power source 32. Controller 10 operates to control the output of pump 8. For embodiments having more than one discreet reservoir, controller 10 operates to control the output of both pumps independently. In yet another embodiment having more than one discreet reservoir, controller 10 controls the mixing of fluid from the reservoirs and the output of the resulting mixture. Such mixing can occur either within or outside of housing 13.

Housing 13 is illustratively a plastic housing that at least partially encloses each of fluid pathway 14, metering solenoid 16, microcontroller 18, sensors 20, 22, buttons 24, 26, 28, indicators 30, and power source 32. Housing 13 is sized to be of similar or smaller size than pump 8 and to be easily transported or worn by a user.

Fluid pathway 14 provides a pathway from input 12 to output 34 of controller 10. Pathway 14 includes metered leg 36 and constant leg 38. Pathway 14 interfaces with sensor 20, which is a pressure sensor. The interface of pathway 14 with sensor 20 allows the pressure being provided by pump 8 to be measured when pump 8 is coupled to and in fluid connection with input 12. Metered leg 36 interfaces with metering solenoid 16. Flow through metered leg 36 is determined by the state of solenoid 16.

Constant leg 38 allows a constant connection and flow between input 12 and output 34. Constant leg 38 includes flow restrictor 40 that is chosen to allow a desired constant flow rate. Alternatively, embodiments are envisioned without constant leg 38. In such embodiments, any desired constant flow rate is provided via metered leg 36 through solenoid 16 as described below. Embodiments with constant leg 38 allow for the setting of a constant base rate via flow restrictor 40. Such embodiments allow the base rate to be administered without drawing power to open solenoid 16. Furthermore, such embodiments continue to administer fluid at times that solenoid 16 is unable to receive power.

Metering solenoid 16 is a normally closed solenoid that is disposed within metered leg 36. A closed position of solenoid 16 prevents flow from input 12 to output 34 via metered leg 36. Similarly, an open position of solenoid 16 allows flow from input 12 to output 34 via metered leg 36. Solenoid 16 is electrically coupled to microcontroller 18 which delivers electrical signals to control the open/closed state of solenoid 16. In the provided embodiment, solenoid 16 has a flow channel that is 0.3″ in diameter and 1.35″ long having a continuous flow rate of 30 ml/hr. The overall throughput (flow rate) of metered leg 36 is controlled and adjusted by electronic pulses received from microcontroller 18. Exemplarily, pulses occur every 30 seconds to open solenoid 16. The time that solenoid 16 remains open during each pulse is varied to achieve the desired flow rate. For example, if the desired flow rate were 10 ml/hr, solenoid 16 would open for 10 seconds, and then close for 20 seconds. Thus, solenoid 16 would be open ⅓ of the time and achieve ⅓ of the continuous flow rate (30 ml/hr) to get 10 ml/hr. The sizes and throughput of solenoid 16 are adjusted to achieve the desired throughput for particular applications chosen by the healthcare provider.

Microcontroller 18 is illustratively a flash programmable microcontroller with onboard memory and ram (not shown). Microcontroller 18 receives input from a plurality of sources 20, 21, 22, 24, 26, 28, 29. The onboard memory stores instructions thereon the instruct microcontroller 18 how to interpret input as part of a regimen program. The program includes a regimen determined by a healthcare provider that allows the operation of solenoid 16 to vary over time according to the instructions in the program. Furthermore, the program includes instructions that allow operation of solenoid 16 that is dependent upon input received from input sources 20, 21, 22, 24, 26, 28, 29 to achieve the output prescribed by the program.

Sensor 20 is a pressure sensor. Some pumps, such as elastomeric balloon reservoir pumps 8 provide an output that varies dependent upon various environmental factors. Such factors include the degree of fullness and expansion of the elastomeric bladder, the ambient atmospheric pressure, ambient temperature, fluid viscosity, and gravity. Sensor 20 senses the pressure of the fluid being provided by pump 8 and supplies that information electronically to microcontroller 18.

Similarly, sensor 22 is a temperature sensor. Sensor 22 electronically provides temperature information to microcontroller 18. Sensor 21 is an accelerometer. Sensor 21 electronically provides information to microcontroller 18 regarding accelerations experienced thereby. Such accelerations provide indication of whether the user wearing controller 10 is engaging in physical activity or in a rest state. Additionally, sensor 21 gives indications of patient position. It should be appreciated that sensor 21 is actually a plurality of sensors in certain embodiments. Accordingly, the program of microcontroller 18 is programmed to provide dosing adjustments based upon the input from sensors 21, 22. Collectively, conditions such as pressure variation (regardless of the source), ambient temperature, and patient activity are able to result in environment induced pressure variations from pump 8 that are translated to solenoid 16. By sensing such conditions, controller 10 is able to account for such environment induced pressure variations and produce a desired flow at output 34. For embodiments having more than one discreet reservoir, controller 10 adjusts the output of each reservoir and the mix ratio based upon input from sensors 20, 21, 22.

Button 24 is a bolus dose button. Pressing button 24 sends a signal to microcontroller 18 that a bolus dose is desired. The program of microcontroller 18 then controls solenoid 16 to effect the bolus dose. It should be appreciated that a caregiver can disable button 24 if desired. Furthermore, embodiments are envisioned where button 24 does not exist.

Buttons 26, 28 are “Flow Rate Up” and “Flow Rate Down” buttons, respectively. Buttons 26, 28 allow for the adjustment of the flow rate through metered leg 36 by the user or a healthcare provider. Again, it should be appreciated that a caregiver can disable buttons 26, 28 if desired. Furthermore, embodiments are envisioned where buttons 26, 28 do not exist.

Electrical I/O port 29 is illustratively a RS-232 port on the outside of housing 13. Port 29 is capable of outputting real-time data. Such data includes a time counter value, pressure sensor reading, temperature sensor reading, flow rate setting, battery voltage, solenoid valve state, and an accumulator count for a bolus. The accumulator count is a timer that is invoked as part of the delivery of a bolus dose. The accumulator provides an indication of the progress of the bolus dose and how much of the bolus dose has been delivered and allows microcontroller 18 to determine when the desired bolus dose has been delivered. Port 29 also allows for controller 10 to be coupled to a computer or other programming device (not shown). Such programming device allows different programs and different settings to be communicated and saved in the flash memory of microcontroller 18. In another embodiment, microcontroller 18 is able to wirelessly transmit data for reporting and programming instead of or in addition to the transmission via port 29.

Indicators 30 are illustratively a plurality of LED's. Embodiments are also envisioned where indicators 30 are one or more screens capable of communicating information to a user and/or health care provider. Indicators 30 are also envisioned as providing audio information. Battery 32 is coupled to microcontroller 18 and powers controller 10. In the provided example, battery 32 is chosen to have enough energy therein to power controller 10 beyond the time it takes to release the dose of fluid provided in pump 18.

In operation, controller 10 is programmed by a healthcare provider to provide a prescribed dosing regimen. In the provided example, the user is prevented from adjusting the dosing regimen, with the exception of allowing bolus dose additions, as will be discussed below. It should be appreciated that embodiments are envisioned where the user/patient is permitted to adjust the dosing regimen. The patient/user is prevented from adjusting the dosing by requiring a computer or other programming device with proper software thereon to be connected via port 29 to configure dosing via an adjustment mode. Alternatively, a specific button 24, 26, 28 combination must be pressed to enter into an adjustment mode. Once in adjustment mode, buttons 26, 28 become active or computer access is achieved to allow an increase or decrease in a steady state flow (such as 10 ml/hr). Confirmation and indication of such increases and decreases are provided to the user via indicators 30.

In addition to a steady state prescriptions, prescriptions that change with time can also be programmed into microcontroller 18. Such prescriptions can decrease over time, increase and decrease along with expected periods of activity and sleep, respectively, or any other variation desired by the healthcare provider. These more detailed prescription profiles are achieved by configuring them on a computer with appropriate software thereon and then interfacing the computer with controller 10. However, embodiments are envisioned where the more detailed prescription profiles are programmed directly on controller 10 using buttons 24, 26, 28.

Part of the program includes what adjustments are to be made to the operation of solenoid 16 to regulate the dose being delivered, if any, when various input is received from sensors 20, sensors 22, sensors 21. By example, if sensors 20 indicates a decreased pressure, due to being at high elevation, a manufacturing variance in pump 8, low fill in pump 8, or for some other reason, microcontroller 18 knows that the base rate of metering solenoid 16 is likely reduced. Accordingly, microcontroller 18 knows to adjust the open time of the duty cycle for a longer time relative to the time it is held open when sensors 20 reads a higher pressure such that controller 10 continues to achieve the desired output at output 34. Similarly, microcontroller 18 adjusts operation of metering solenoid 16 in response to input received from sensors 22. Also similarly, microcontroller 18 adjusts operation of metering solenoid 16 in response to input received from sensors 21. For uses of controller 10 as a provider of post surgical treatment, activity by a patient, which would register with sensors 21, can cause increased pain and increased swelling. Accordingly, microcontroller 18 is programmed to provide an increased dose of fluid, illustratively an anesthetic or anti-inflammatory, in response to the sensing of activity via sensors 21.

The program within microcontroller 18 is also able to enable or disable bolus button 24. When enabled, a patient/user presses bolus button 24 whenever a bolus dose of fluid is desired to be administered. The program can dictate how large of a bolus dose is administered in response to activation of button 24. Additionally, the program dictates a lock out period after administration of a bolus dose in which another bolus dose can not be administered. Embodiments are envisioned where there is no lock out period. In the provided example, a bolus dose is achieved by holding metering solenoid 16 open for a period of time such that the bolus amount is allowed to pass therethrough and out of output 34. Again, the time period to achieve the bolus amount takes into account information received from sensors 20, 22, 21.

Additionally, embodiments are envisioned where the sensors 20 provides updates to microcontroller 18 every fixed segment of time, such as three seconds. Furthermore, embodiments exist where sensors 20 only takes readings and provides information to microcontroller 18 when metering solenoid 16 is in a closed position so as to provide only a static pump output pressure as opposed to a fluctuating pressure resulting from the outflowing of fluid through an open metering solenoid 16.

The programming of microcontroller 18 also allows controller 10 to be self proving. In other words, microcontroller 18 can monitor the performance of controller 10 and confirm that it is operating as desired. Furthermore, controller 10 can provide an operation status to a user/patient or healthcare professional via indicators 30.

While the above controller 10 has been described as a disposable controller having solenoid 16 that comes into direct contact with the fluid to be dispensed, the concepts and pieces can be utilized as part of a reusable controller as well. In such embodiments, only pumps 8, and tubing sets that form the pathways within controller 10, connections upstream of controller 10 between pump 8 and controller 10, and downstream of controller 10 are disposable. Controller 10, including, solenoid 16, microcontroller 18, and power source 32, is able to be returned, checked, and reused. One embodiment of reusable controller 10 includes solenoid 16 powering a pinch valve that selectively pinches off flow through metered leg 36. One such pinch valve is the PV256 made by Instech laboratories, Inc. Disposable tubing is placed within the pinch valve such that pinching by the pinch valve is translated to the tubing to selectively cut off flow therethrough. Accordingly, any piece that would come into contact with the fluid medication is able to be changed out between uses. One embodiment of tubing sets for use with reusable controller 10 includes one or more sensors (e.g. pressure sensor 20 and temperature sensor 22) integrated therein with an electrical lead extending therefrom for connection to microcontroller 18 through a plug or otherwise. Furthermore, one embodiment of reusable controller 10 employs power source 32 that is a rechargeable battery. Microcontroller 18 in reusable controller 18 is programmed to monitor how many cycles controller 18 has gone through. By so monitoring, the cycle values can be reported out and can indicate when servicing should be undertaken or when reusable controller 10 should be retired.

Although the disclosure has been described in detail with reference to certain preferred embodiments, variations and modifications exist within the spirit and scope of the disclosure as described and defined in the following claims.

1. A portable kit for providing pain relief comprising:
a catheter for delivering an anesthetic-containing fluid adjacent a wound site in a body;
tubing attached to the catheter and having a lumen in fluid communication with a lumen of the catheter;
a pump adapted to deliver fluid, the pump including an outlet in fluid communication with the tubing, the pump further including an inlet port for filling a reservoir of the pump; the pump affixable to the body to permit portability;
an electronic valve disposed between the catheter and the reservoir in a first flow path;
a controller that generates signals to control operation of the electronic valve; and
instructions on a controller interpretable medium that when interpreted by the controller cause the controller to be able to selectively place the electronic valve in each of three conditions, the first condition causing the electronic valve to periodically open and close to achieve a first output flow rate through the catheter, a second condition causing the electronic valve to open and close to achieve a second output flow rate, and the third condition causing closure of the electronic valve.

2. The kit of claim 1, wherein the pump is non-powered.

3. The kit of claim 1, wherein the valve is in continuous fluid communication with the pump reservoir.

4. The kit of claim 1, further including a patient accessible actuator, activation of the actuator causing the controller to generate signals instructing the electronic valve to assume the second condition.

5. The kit of claim 1, further including a second flow path in parallel with the first flow path such that anesthetic may travel from the reservoir to the catheter without passing through the electronic valve, the second flow path including a flow restrictor therein.

6. The kit of claim 1, wherein the second condition is achieved by causing the electronic valve to open for longer periods of time relative to the first condition.

7. The kit of claim 1, wherein the second condition is achieved by causing the electronic valve to open more often relative to the first condition.

8. A portable kit for providing pain relief comprising:
a first pump having a first reservoir that when filled with an anesthetic provides a first pressure biased to evacuate the anesthetic from the first pump; the first pump being constructed such that the first pressure varies based upon the first reservoir experiencing environment induced pressure variations; and
an electronic controller that controls the evacuation of anesthetic from the first reservoir to compensate for the variability in the first pressure to achieve a desired output flow that is independent of the environment induced pressure variations experienced by the first reservoir.

9. The kit of claim 8, wherein the controller further controls the evacuation to compensate for variability in at least one of anesthetic viscosity, pump pressure, patient activity, temperature, reservoir fullness, and patient position.

10. The kit of claim 8, further including a second pump having a second reservoir, the electronic controller also controlling evacuation of fluid from the second reservoir.

11. The kit of claim 10, wherein the output of the first pump is mixed with the output of the second pump, the electronic controller controlling the outputs of each pump to control the mixture percentage resulting from the mixing of the outputs of the first and second pumps.

12. The kit of claim 11, wherein the controller adjusts the mixture percentage based on at least one of temperature, pressure, fluid viscosity, patient activity, and patient position experienced by at least one of the pumps.

13. A portable kit for providing medication to tissue comprising:
a pump having a first reservoir, the first reservoir, when filled with a first fluid provides a first pressure biased to evacuate the first fluid from the first reservoir; and
an electronic controller that controls the evacuation of fluid from the first reservoir, the electronic controller being able to receive input indicative of the activity of a patient, the electronic controller adjusting an output flow from the first reservoir at least partially based upon the input indicative of the activity of the patient.

14. The kit of claim 13, wherein the electronic controller further adjusts the output flow from the first reservoir at least partially based upon an input indicative of the position of the patient.

15. The kit of claim 13, further including a second reservoir for containing a second fluid such that the first and second fluids are mixed in first ratio for a first period of time by the electronic controller, and the first and second fluids are mixed in a second ratio for a second period of time by the electronic controller.

16. The kit of claim 15, wherein the controller determines the first and second ratios and the first and second periods of time based upon the input indicative of the activity of the patient.

17. The kit of claim 15, wherein the first and second ratios and the first and second periods of time are configured to effect a programmable regimen having a changing fluid delivery rate and composition.

18. A portable kit for providing fluid to tissue comprising:
a pump having a first reservoir and a second reservoir,
an electronic controller that controls the evacuation of fluid from the first and second reservoirs, the electronic controller being able to receive input selected from the group including input indicative of patient status and input indicative of atmospheric status, the electronic controller adjusting output flows from the first and second reservoirs at least partially based upon the received input to achieve a output mixture of the first and second fluids, the mixture having a first ratio of the first fluid to the second fluid.

19. The kit of claim 18, wherein the first and second reservoirs, when filled with a fluid provide first and second pressures, respectively, biased to evacuate the fluid from the first and second reservoirs.

20. The kit of claim 18, wherein the controller adjusts the output flows of the first and second fluids upon receiving a change in the inputs such that the mixture has a second ratio of the first fluid to the second fluid.

21. A portable kit for providing fluid to tissue comprising:
a pump having a first reservoir and a second reservoir,
an electronic controller that controls the evacuation of fluid from the first and second reservoirs, the electronic controller being programmable to cause an output flow of the pump to vary as a function of time.

22. The kit of claim 21, wherein the controller is further able to vary both the absolute and the relative amount of fluid expelled from each of the reservoirs over time.

23. The kit of claim 21, wherein the controller is programmed to vary the output flow based upon input received from sensors.
